(19) United States
(12) Patent Application Publication
(10) Pub. No.: US 2003/0165818 A1
(43) Pub. Date: Sep. 4, 2003

(54) Novel protein

(76) Inventors: Mark Robert Edbrooke, Stevenage (GB); Alan Peter Lewis, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/054,776

(22) Filed: Jan. 23, 2002

(30) Foreign Application Priority Data
Jan. 23, 2001 (GB) 1.0101760.7

(51) Int. Cl. C12Q 1/70; C12Q 1/37

(52) U.S. Cl. 435/5; 435/23

(57) ABSTRACT

The present invention provides a method for identification of a substance that modulates serine protease activity, which method comprises:

(i) contacting

(a) a cell selected from a neuronal cell, a lung cell, an intestinal cell and a cell infected with a virus, which cell expresses a serine protease polypeptide comprising the amino acid sequence of SEQ ID NO: 2 or a variant thereof which has dipeptidyl peptidase activity with

(b) a test substance; and

(ii) monitoring for serine protease activity.

### FIELD OF THE INVENTION

The present invention relates to serine protease polypeptides.

### BACKGROUND OF THE INVENTION

There are 4 main catalytic types of peptidases; serine (which includes threonine peptidases) cysteine, aspartic and metallo. The serine, threonine and cysteine peptidases are catalytically very different from the aspartic and metallopeptidases in that the nucleophile of the catalytic site is part of an amino acid, whereas it is an activated water molecule in the other groups.

Serine peptidases are enzymes in which the catalytic mechanism depends upon the hydroxyl group of a serine residue acting as the nucleophile that attacks the peptide bond. Examples of serine peptidases include trypsin, chymotrypsin, thrombin, plasmin, furin, cathepsin G, chymase, tryptase and granzymes A, B, K and M. Threonine-dependent peptidases can be grouped with the serine peptidases and include the proteasome.

The dipeptidyl peptidase (DPP) family is, under the MEROPS protease database, classified as Clan SC and Family S9. Clan SC contains peptidase families in which the catalytic triad has been identified as being serine, aspartate, histidine. The Family S9 includes prolyl oligopeptidase, acylaminoacylpeptidase and DPP IV. Although these peptidases are very divergent in structure, and different in catalytic activity, they all possess C-terminal catalytic domains with significant sequence similarity.

Subfamily S9C includes DPP IV, examples of which are known in both bacteria and eukaryotes. DPP IV is a serine exopeptidase that cleaves Xaa-Pro dipeptides from the N-terminus of oligo- and polypeptides. The proline residue can be substituted by alanine or hydroxyproline, although this decreases the rate of hydrolysis. The identity of the N-terminal residue is not important for enzymic activity, though it must have a free amino group. DPP IV cannot hydrolyse substrates with proline or hydroxyproline in the P1′ position.

The tertiary structure of DPP IV contains an N-terminal seven-bladed β propeller domain and a C-terminal α/β hydrolase fold (domain).

DPP IV is a type II membrane protein with an N-terminal hydrophobic sequence representing an uncleavable signal peptide that also acts as a membrane-anchoring domain. DPP IV has a short cytoplasmic tail (6 residues), a transmembrane domain (23 residues) and a long extracellular domain (738 residues).

### SUMMARY OF THE INVENTION

The present invention is based on the novel finding that expression of that a serine protease, referred to herein as HIPHUM 46, is up-regulated or down-regulated in various neuronal diseases and following viral infection. HIPHUM 46 is up-regulated in frontal cortex from Alzheimer's, myotonic dystrophy, parasupranuclear palsy (PSP), Huntington's and amyotrophic lateral sclerosis (ALS) brain, in parietal cortex from PSP, Huntington's and ALS brain, in bFGF treated endothelial cells, PBMC's and in HBV infection. HIPHUM 46 is down-regulated in cerebellum from Alzheimer's brain, in HIV infection and in HSV infection. In healthy tissue, HIPHUM 46 is shown to be primarily expressed in cerebellum, jejunum, lung and rectum. HIPHUM 46 is a screening target for the identification and development of novel pharmaceutical agents, including modulators of serine protease activity which may be used in the treatment and/or prophylaxis of disorders such as musculoskeletal diseases including osteoarthritis, HBV diseases, Alzheimer's disease and other diseases of the central nervous system including parasupranuclear palsy, myotonic dystrophy, Huntington's disease and amyotrophic lateral sclerosis. HIPHUM 46 and variants and fragments thereof, HIPHUM 46 polynucleotides and HIPHUM 46 antibodies may also be used in the treatment of these diseases.

Accordingly, the present invention provides:

a method for identification of a substance that modulates serine protease activity, which method comprises:

(i) contacting

(a) a cell selected from a neuronal cell, a lung cell, an intestinal cell and a cell infected with a virus, which cell expresses a serine protease polypeptide comprising the amino acid sequence of SEQ ID NO: 2 or a variant thereof which has dipeptidyl peptidase activity; and

(b) a test substance; and

(ii) monitoring for serine protease activity;

a method for identification of a substance that modulates serine protease activity, which method comprises:

(i) contacting

(a) a serine protease polypeptide isolated from a cell selected from a neuronal cell, a lung cell, an intestinal cell and a cell infected with a virus, which serine protease polypeptide comprises the amino acid sequence of SEQ ID NO: 2 or a variant thereof which has dipeptidyl peptidase activity; and

(b) a test substance; and

(ii) monitoring for serine protease activity;

a modulator of serine protease activity identified by a method according to the invention for use in a method of treatment of the human or animal body by therapy.

a modulator of serine protease activity identified by a method according to the invention in the manufacture of a medicament for the treatment of a muscloskeletal disease, a HBV disease, Alzheimer's disease, parasupranuclear palsy, myotonic dystrophy, Huntington's disease or amyotrophic lateral sclerosis.

use of a serine protease polypeptide comprising

(i) the amino acid sequence of SEQ ID NO: 2; or

(ii) a variant thereof which has dipeptidyl peptidase activity; or

(iii) a fragment of (i) or (ii) which has dipeptidyl peptidase activity in the manufacture of a medicament for use in the treatment of a musculoskeletal disease, a HBV disease, Alzheimer's disease, parasupranuclear palsy, myotonic dystrophy, Huntington's disease or amyotrophic lateral sclerosis;

use of a polynucleotide encoding a serine protease polypeptide comprising

(i) the amino acid sequence of SEQ ID NO: 2; or

(ii) a variant thereof which has dipeptidyl peptidase activity; or

(iii) a fragment of (i) or (ii) which has dipeptidyl peptidase activity in the manufacture of a medicament for use in the treatment of a musculoskeletal disease, a HBV disease, Alzheimer's disease, parasupranuclear palsy, myotonic dystrophy, Huntington's disease or amyotrophic lateral sclerosis.

use of an antibody specific for a serine protease polypeptide comprising the amino acid sequence of SEQ ID NO: 2 in the manufacture of a medicament for use in the treatment of a musculoskeletal disease, a HBV disease, Alzheimer's disease, parasupranuclear palsy, myotonic dystrophy, Huntington's disease or amyotrophic lateral sclerosis;

use of a substance which modulates proteinase activity of a polypeptide with the amino acid sequence shown in SEQ ID NO: 2 or a variant thereof in the manufacture of a medicament for treatment or prophylaxis a musculoskeletal disease, a HBV disease, Alzheimer's disease, parasupranuclear palsy, myotonic dystrophy, Huntington's disease or amyotrophic lateral sclerosis;

a method of treating a subject suffering from a musculoskeletal disease, a HBV disease, Alzheimer's disease, parasupranuclear palsy, myotonic dystrophy, Huntington's disease or amyotrophic lateral sclerosis, which method comprises administering to said subject an effective amount of a substance which modulates proteinase activity of a polypeptide with the amino acid sequence shown in SEQ ID NO: 2 or a variant thereof;

a method for treating a musculoskeletal disease, a HBV disease, Alzheimer's disease, parasupranuclear palsy, myotonic dystrophy, Huntington's disease or amyotrophic lateral sclerosis, which method comprises:

(i) identifying a substance that modulates serine protease activity and/or expression

(ii) administering an effective amount of the substance to a patient suffering from a musculoskeletal disease, a HBV disease, Alzheimer's disease, parasupranuclear palsy, myotonic dystrophy, Huntington's disease or amyotrophic lateral sclerosis.

### BRIEF DESCRIPTION OF THE FIGURES

FIG. 1 shows the relative expression levels of HIPHUM46 in various normal tissues.

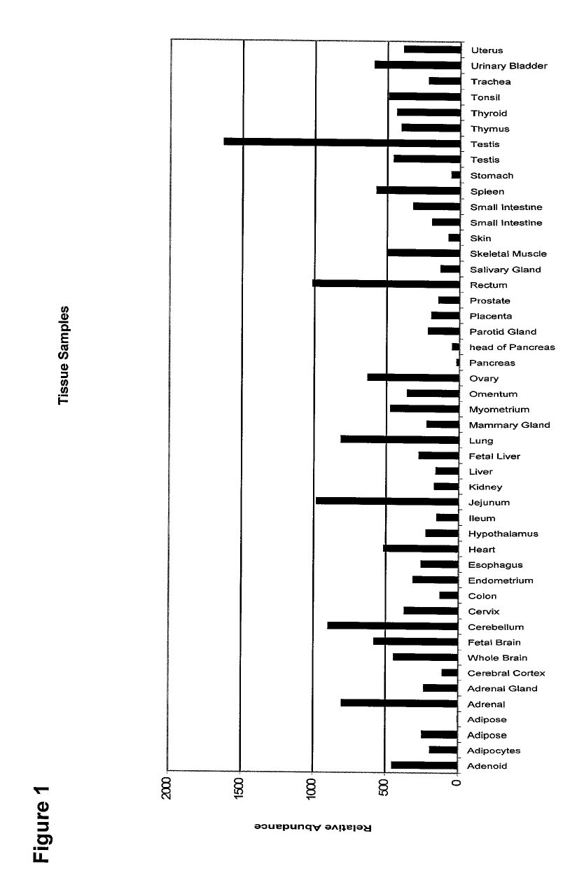

FIG. 2 shows the relative expression levels in normal brain, Alzheimer's disease brain (Alz.), myotonic dystrophy brain (MD), parasupranuclear palsy brain (PSP), Huntington's disease brain (Hunt.) and amyotrophic lateral sclerosis brain (ALS). FIG. 2 also shows the relative expression levels in normal lung, asthma lung and chronic obstructive pulmonary lung (COPD).

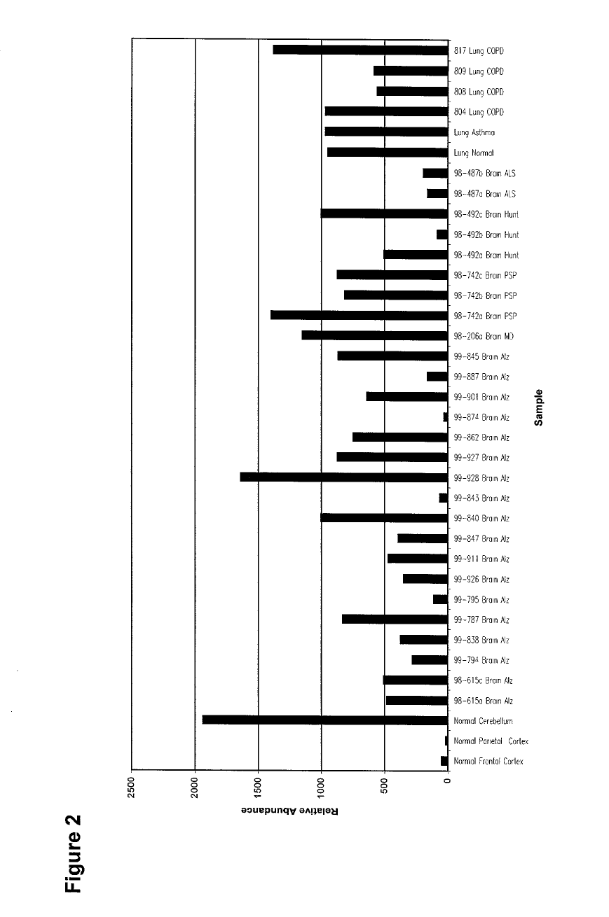

FIG. 3 shows the relative expression levels in various cells of the immune system, in stimulated and unstimulated bone marrow, in normal and osteoarthritic (OA) knee cartilage, in differentiated and undifferentiated osteoblasts, in rheumatoid arthritis (RA) and in various viral infections.

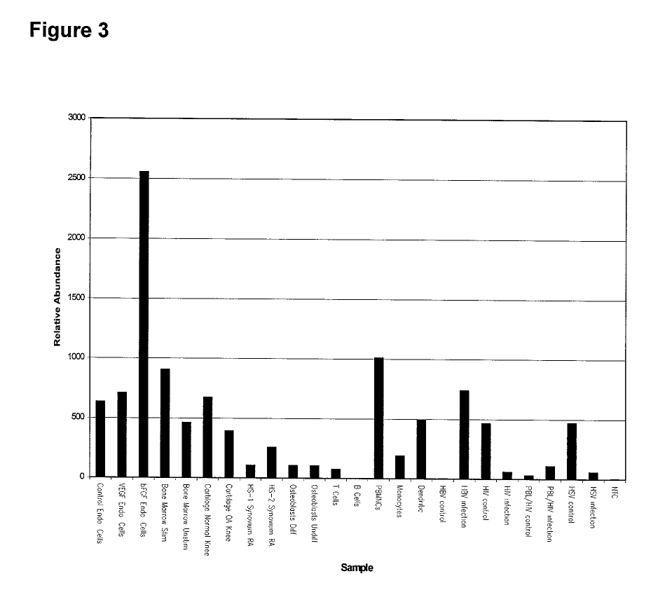

### BRIEF DESCRIPTION OF THE SEQUENCES

SEQ ID NO: 1 shows the nucleotide and amino acid sequences of human protein HIPHUM 46.

SEQ ID NO: 2 is the amino acid sequence alone of HIPHUM 46.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to the use of a human serine protease, referred to herein as HIPHUM 46, and variants and fragments thereof. Sequence information for HIPHUM 46 is provided in SEQ ID NO: 1 (nucleotide and amino acid) and in SEQ ID NO: 2. A polypeptide of the invention thus consists essentially of the amino acid sequence of SEQ ID NO: 2 or of a variant of that sequence, or of a fragment of either thereof.

The term “variant” refers to a polypeptide which has a same essential character or basic biological functionality as HIPHUM 46. The essential character of HIPHUM 46 can be defined as follows: HIPHUM 46 is a serine protease. Preferably the polypeptide has dipeptidyl peptidase activity. Preferably a variant polypeptide is one which cleaves the same substrate as HIPHUM 46. A suitable substrate peptide typically comprises a p-nitrophenylanilide (pNA) which, when hydrolyzed by HIPHUM 46, releases the absorbant p-nitrophenylaniline. A typical substrate contains the P4-P1 residues capped on the C-terminus with either pNA or 7-amino-4-trifluromethylcoumarin (AMC), though dipeptidases may only require P2-P1. Typical substrates include: H-Ala-Pro-pNA, H-Gly-Pro-pNA, Gly-Pro-AMC and H-Arg-Pro-pNA.

Typically a variant polypeptide comprises a peptidase S9 domain. This domain is conserved in the S9 or prolyl oligopeptidase family, which consists of a number of evolutionary related serine peptidases whose catalytic activity seems to be provided by a charge relay system similar to that of the trypsin family, but which evolved by independent convergent evolution. Preferably a variant polypeptide comprises catalytic residues of serine, aspartate and histidine residues at positions corresponding to positions 739, 817, 849 of SEQ ID NO: 2.

A variant polypeptide typically comprises a DPP IV N-terminal domain. This domain is found to the N-terminal side of the active site of dipeptidyl aminopeptidases related to dipeptidyl peptidase IV (DPP IV). A variant polypeptide typically comprises a DPP IV β propeller domain and/or a α/β hydrolase domain. A variant polypeptide preferably comprises paired glutamates of the β propeller domain at positions corresponding to residues 259, 260 of SEQ ID NO: 2.

A polypeptide having the same essential character as HIPHUM 46 may be identified by monitoring for dipeptidyl peptidase activity.

In another aspect of the invention, a variant is one which does not show the same activity as HIPHUM 46 but is one which inhibits a basic function of HIPHUM 46. For example, a variant polypeptide is one which inhibits peptidase activity of HIPHUM 46, for example by binding to substrate to prevent cleavage of the substrate by HIPHUM 46.

Typically, polypeptides with more than about 65% identity preferably at least 80% or at least 90% and particularly preferably at least 95% at least 97% or at least 99% identity, with the amino acid sequences of SEQ ID NO: 2, are considered as variants of the proteins. Such variants may include allelic variants and the deletion, modification or addition of single amino acids or groups of amino acids within the protein sequence, as long as the peptide maintains the basic biological functionality of HIPHUM 46.

Amino acid substitutions may be made, for example from 1, 2 or 3 to 10, 20 or 30 substitutions. The modified polypeptide generally retains activity as a serine protease. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Shorter polypeptide sequences are within the scope of the invention. For example, a peptide of at least 20 amino acids or up to 50, 60, 70, 80, 100, 150, 200, 300 or 400 amino acids in length is considered to fall within the scope of the invention as long as it demonstrates a basic biological functionality of HIPHUM 46. In particular, but not exclusively, this aspect of the invention encompasses the situation when the protein is a fragment of the complete protein sequence and may represent a catalytic domain or substrate binding domain. Preferred fragments include fragments comprising a S9 peptidase domain. Such fragments can be used to construct chimeric proteases preferably with another protease, more preferably with another member of the family of serine proteases. Such chimeric proteases may comprise different domains from different serine proteases. For example, a fragment comprising a S9 peptidase domain of a polypeptide of the invention may be fused to a N-terminal domain of a different serine protease.

Fragments of HIPHUM 46 or a variant thereof can also be used to raise anti-HIPHUM 46 antibodies which may be used in the manufacture of a medicament for use in the treatment of musculoskeletal diseases including osteoarthritis, HBV diseases, Alzheimer's disease, other diseases of the central nervous system including parasupranuclear palsy, myotonic dystrophy, Huntington's disease and amyotrophic lateral sclerosis, malabsorption syndromes, irritable bowel syndrome, lung disease, type-I diabetes, fecal incontinence, hemorrhoids, proctitis, rectal polyps, small bowel tumors, colorectal tumors, anemia, dyslexia, ceroid lipofuscinosis, allergic encephalomyelitis and multiple sclerosis. The antibody may be produced in response to a fragment of HIPHUM 46 which comprises an epitope of the HIPHUM 46 polypeptide which may not demonstrate the catalytic, substrate binding or other properties of HIPHUM 46.

Polypeptides for use in the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

Nucleotide sequences that encode for HIPHUM 46 or variants thereof as well as nucleotide sequences which are complementary thereto may be used in a method of the invention. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al, 1989.

Typically a polynucleotide for use in the invention comprises a contiguous sequence of nucleotides which is capable of hybridizing under selective conditions to the coding sequence or the complement of the coding sequence of SEQ ID NO: 1.

A polynucleotide for use in the invention can hydridize to the coding sequence or the complement of the coding sequence of SEQ ID NO: 1 at a level significantly above background. Background hybridization may occur, for example, because of other cDNAs present in a cDNA library. The signal level generated by the interaction between a polynucleotide for use in the invention and the coding sequence or complement of the coding sequence of SEQ ID NO: 1 is typically at least 10 fold, preferably at least 100 fold, as intense as interactions between other polynucleotides and the coding sequence of SEQ ID NO: 1. The intensity of interaction may be measured, for example, by radiolabelling the probe, e.g. with ³²P. Selective hybridisation may typically be achieved using conditions of medium to high stringency. However, such hybridisation may be carried out under any suitable conditions known in the art (see Sambrook et al, 1989. For example, if high stringency is required suitable conditions include from 0.1 to 0.2×SSC at 60° C. up to 65° C. If lower stringency is required suitable conditions include 2×SSC at 60° C.

The coding sequence of SEQ ID NO: 1 may be modified by nucleotide substitutions, for example from 1, 2 or 3 to 10, 25, 50 or 100 substitutions. The polynucleotide of SEQ ID NO: 1 may alternatively or additionally be modified by one or more insertions and/or deletions and/or by an extension at either or both ends. A polynucleotide may include one or more introns, for example may comprise genomic DNA. Additional sequences such as signal sequences which may assist in insertion of the polypeptide in a cell membrane may also be included. The modified polynucleotide generally encodes a polypeptide which has a HIPHUM 46 activity. Alternatively, a polynucleotide encodes a catalytic or substrate-binding portion of a polypeptide or a polypeptide which inhibits HIPHUM 46 activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 1 over a region of at least 20, preferably at least 30, for instance at least 40, at least 60, more preferably at least 100 contiguous nucleotides or most preferably over the full length of SEQ ID NO: 1.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides for use in the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides may be utilised in gene therapy techniques. Nucleotides complementary to those encoding HIPHUM 46, or antisense sequences, may also be used in gene therapy.

The present invention also includes the use of expression vectors that comprise nucleotide sequences encoding the HIPHUM 46 or variants thereof in a method of the invention or in the manufacture of a medicament for use in the treatment of musculoskeletal diseases including osteoarthritis, HBV diseases, Alzheimer's disease, other diseases of the central nervous system including parasupranuclear palsy, myotonic dystrophy, Huntington's disease and amyotrophic lateral sclerosis, malabsorption syndromes, irritable bowel syndrome, lung disease, type-I diabetes, fecal incontinence, hemorrhoids, proctitis, rectal polyps, small bowel tumors, colorectal tumors, anemia, dyslexia, ceroid lipofuscinosis, allergic encephalomyelitis and multiple sclerosis. Expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. 1989.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used in a method treatment of musculoskeletal diseases including osteoarthritis, HBV diseases, Alzheimer's disease, other diseases of the central nervous system including parasupranuclear palsy, myotonic dystrophy, Huntington's disease and amyotrophic lateral sclerosis, malabsorption syndromes, irritable bowel syndrome, lung disease, type-I diabetes, fecal incontinence, hemorrhoids, proctitis, rectal polyps, small bowel tumors, colorectal tumors, anemia, dyslexia, ceroid lipofuscinosis, allergic encephalomyelitis and multiple sclerosis.

Preferably, a polynucleotide for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid or virus vectors provided with a origin of replication, optionally a promoter for the expression of the said polynucleotide and optionally a regulator of the promoter. Vectors may be used in vitro, for example to transfect cells which have been removed from a patient. The transfected cells may then be returned to the patient. Vectors may also be adapted to be used in vivo, for example in a method of gene therapy.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences. This will allow the introduction of the polynucleotides of the invention into the genome of eukaryotic cells by homologous recombination. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The invention also includes cells that have been modified to express the HIPHUM 46 polypeptide or a variant thereof. Such cells may be used in a method of the invention or in a method of treatment of musculoskeletal diseases including osteoarthritis, HBV diseases, Alzheimer's disease, other diseases of the central nervous system including parasupranuclear palsy, myotonic dystrophy, Huntington's disease and amyotrophic lateral sclerosis, malabsorption syndromes, irritable bowel syndrome, lung disease, type-I diabetes, fecal incontinence, hemorrhoids, proctitis, rectal polyps, small bowel tumors, colorectal tumors, anemia, dyslexia, ceroid lipofuscinosis, allergic encephalomyelitis and multiple sclerosis or in the manufacture of a medicament for the treatment of any such disease.

According to another aspect, the present invention also relates to the use of antibodies specific for a HIPHUM 46 or a variant thereof in a method of treatment of musculoskeletal diseases including osteoarthritis, HBV diseases, Alzheimer's disease, other diseases of the central nervous system including parasupranuclear palsy, myotonic dystrophy, Huntington's disease and amyotrophic lateral sclerosis, malabsorption syndromes, irritable bowel syndrome, lung disease, type-I diabetes, fecal incontinence, hemorrhoids, proctitis, rectal polyps, small bowel tumors, colorectal tumors, anemia, dyslexia, ceroid lipofuscinosis, allergic encephalomyelitis and multiple sclerosis or in the manufacture of a medicament for the treatment of any such disease.

Antibodies may be raised against specific epitopes of the polypeptides according to the invention. Such antibodies may be used to block substrate binding to the receptor. An antibody, or other compound, “specifically binds” to a protein when it binds with preferential or high affinity to the protein for which it is specific but does substantially bind not bind or binds with only low affinity to other proteins. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

Antibodies for use in the invention may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample, which method comprises:

I providing an antibody of the invention;

II incubating a biological sample with said antibody under conditions which allow for the formation of an antibody-antigen complex; and

III determining whether antibody-antigen complex comprising said antibody is formed.

A sample may be for example a tissue extract, blood, serum and saliva. Antibodies of the invention may be bound to a solid support and/or packaged into kits in a suitable container along with suitable reagents, controls, instructions, etc. Antibodies may be linked to a revealing label and thus may be suitable for use in methods of in vivo HIPHUM 46 imaging.

Antibodies for use in the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An important aspect of the present invention is the use of polypeptides according to the invention in screening methods to identify modulators of HIPHUM 46 activity. Such modulators may be used in a method of treatment of musculoskeletal diseases including osteoarthritis, HBV diseases, Alzheimer's disease, other diseases of the central nervous system including parasupranuclear palsy, myotonic dystrophy, Huntington's disease and amyotrophic lateral sclerosis, malabsorption syndromes, irritable bowel syndrome, lung disease, type-I diabetes, fecal incontinence, hemorrhoids, proctitis, rectal polyps, small bowel tumors, colorectal tumors, anemia, dyslexia, ceroid lipofuscinosis, allergic encephalomyelitis and multiple sclerosis or in the manufacture of a medicament for the treatment of any such disease. The screening methods may be used to identify substances that bind to serine protease and in particular which bind to HIPHUM 46 such as a substrate for the enzyme. Screening methods may also be used to identify agonists or antagonists which may modulate serine protease activity, inhibitors or activators of HIPHUM 46 activity, and/or agents which up-regulate or down-regulate HIPHUM 46 expression.

Any suitable format may be used for the assay. In general terms such screening methods may involve contacting a polypeptide of the invention with a test substance and monitoring for binding of the test substance to the polypeptide or measuring protease activity. A polypeptide of the invention may be incubated with a test substance. Modulation of serine protease activity may be determined. In a preferred aspect, the assay is a cell-based assay. The cell is preferably a neuronal cell, a lung cell, an intestinal cell or a virally infected cell. Preferably the neuronal cell is from the cerebellum or is a cell from a subject suffering from a central nervous system disorder in which expression of HIPHUM 46 is up-regulated or from an animal model of a central nervous system disorder in which expression of HIPHUM 46 is up-regulated. Preferably the virally infected cell is infected with a virus that promotes the up-regulation of HIPHUM 46, such as HBV. Alternatively HIPHUM 46 may be isolated from any such cell. Preferably the assay may be carried out in a single well of a microtitre plate. Assay formats which allow high throughput screening are preferred.

A typical assay for determining whether a test substance acts as an inhibitor or activator of HIPHUM 46 activity comprises monitoring cleavage of a peptide-based substrate using a FRET, HTRF (or TRET) and/or SPA assay. Other assays that may be used to determine whether a test substance acts as an inhibitor or activator of HIPHUM 46 activity include ELISA, zymography, activation or a reporter protease, assays with chimeric proteins and assays with peptides where cleavage is detected by HPLC/LC/MS. Cell based assays may also be used.

A typical peptide-based substrate contains p-nitrophenylanilide (pNA). When hydrolyzed by HIPHUM 46 the substrate releases absorbant p-nitrophenylaniline. A typical substrate contains the P4-P 1 residues capped on the C-terminus with either pNA or 7-amino-4-trifluromethylcoumarin (AMC), though dipeptidases may only require P2-P1. Typical substrates include H-Ala-Pro-pNA.HCl, H-Gly-Pro-pNA.HCl and Gly-Pro-AMC.

Modulator activity can be determined by contacting cells expressing HIPHUM 46 or a variant or fragment thereof with a substance under investigation and by monitoring an effect mediated by the polypeptide. The cells expressing the polypeptide may be in vitro or in vivo. The polypeptide of the invention may be naturally or recombinantly expressed. Preferably, the assay is carried out in vitro using cells expressing recombinant polypeptide. Preferably, control experiments are carried out on cells which do not express the polypeptide of the invention to establish whether the observed responses are the result of activation of the polypeptide.

The binding of a test substance to HIPHUM 46 or a variant of fragment thereof can be determined directly. For example, a radiolabelled test substance can be incubated with the polypeptide of the invention and binding of the test substance to the polypeptide can be monitored. Typically, the radiolabelled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by carrying out a competitive binding assay.

Substances that inhibit the interaction of a polypeptide of the invention with a HIPHUM 46 substrate or with another protease may also be identified through a yeast 2-hybrid assay or other protein interaction assay such as a co-immunoprecipitation or an ELISA based technique.

Assays may be carried out using cells expressing HIPHUM 46, and incubating such cells with the test substance optionally in the presence of a HIPHUM 46 substrate. The results of the assay are compared to the results obtained using the same assay in the absence of the test substance. Cells expressing HIPHUM 46 constitutively may be provided for use in assays for HIPHUM 46 function. Additional test substances may be introduced in any assay to look for inhibitors or activators of substrate binding or inhibitors or activators of protease activity.

Assays may also be carried out to identify substances which modify HIPHUM 46 expression, for example substances which up- or down-regulate expression. Such assays may be carried out for example by using antibodies for HIPHUM 46 to monitor levels of HIPHUM 46 expression. Other assays which can be used to monitor the effect of a test substance on HIPHUM 46 expression include using a reporter gene construct driven by the HIPHUM 46 regulatory sequences as the promoter sequence and monitoring for expression of the reporter polypeptide.

Additional control experiments may be carried out.

Suitable test substances which can be tested in the above assays include combinatorial libraries, defined chemical entities and compounds, peptide and peptide mimetics, oligonucleotides and natural product libraries, such as display (e.g. phage display libraries) and antibody products.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Fragments of HIPHUM 46 which do not show HIPHUM 46 peptidase activity may also be screened to monitor their effect on the activity of full length HIPHUM 46 or a functional variant or fragment thereof.

Test substances may be used in an initial screen of, for example, 10 substances per reaction, and the substances of these batches which show inhibition or activation tested individually. Test substances may be used at a concentration of from 1 nM to 10 mM, preferably from, 100 nM to 1000 μM or from 1 μM to 100 μM, more preferably from 1 μM to 10 μM. Preferably, the activity of a test substance is compared to the activity shown by a known activator or inhibitor. A test substance which acts as an inhibitor may produce a 50% inhibition of activity of the protease. Alternatively a test substance which acts as an activator may produce 50% of the maximal activity produced using a known activator.

Another aspect of the present invention is the use of the substances that have been identified by screening techniques referred to above in the treatment of disease states, which are responsive to regulation of serine protease activity. The treatment may be therapeutic or prophylactic. The condition of a patient suffering from such a disease state can thus be improved.

In particular, such substances may be used in the treatment of musculoskeletal diseases including osteoarthritis, HBV diseases, Alzheimer's disease and other diseases of the central nervous system including parasupranuclear palsy, myotonic dystrophy, Huntington's disease and amyotrophic lateral sclerosis.

Additional disease states that may be treated include malabsorption syndromes, irritable bowel syndrome, lung disease, type-I diabetes, fecal incontinence, hemorrhoids, proctitis, rectal polyps, small bowel tumors, colorectal tumors, anemia, dyslexia, ceroid lipofuscinosis, allergic encephalomyelitis and multiple sclerosis.

Substances that act as inhibitors of HIPHUM 46 activity may be used in the treatment of disease states in which HIPHUM 46 expression is up-regulated such as Alzheimer's disease, myotonic dystrophy (MD), parasupranuclear palsy (PSP), Huntington's disease and amyotrophic lateral sclerosis (ALS) and HBV infection. Substances that activate HIPHUM 46 activity may be used in the treatment of disease states in which expression of HIPHUM 46 is down-regulated such as Alzheimer's disease, HIV infection and HSV infection.

Substances identified according to the screening methods outlined above and polpypeptides, polynucleotides, antibodies and expression vectors described herein may be formulated with standard pharmaceutically acceptable carriers and/or excipients as is routine in the pharmaceutical art. For example, a suitable substance may be dissolved in physiological saline or water for injections. The exact nature of a formulation will depend upon several factors including the particular substance to be administered and the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The substances, polypeptides, polynucleotides, antibodies and expression vectors may be administered by enteral or parenteral routes such as via oral, buccal, anal, pulmonary, intravenous, intra-arterial, intramuscular, intraperitoneal, topical or other appropriate administration routes.

A therapeutically effective amount of a modulator, polpypeptide, polynucleotide, antibody or expression vector is administered to a patient. The dose may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Nucleic acid encoding HIPHUM 46 or a variant thereof which inhibits HIPHUM 46 activity may be administered to the mammal. In particular, a nucleic acid encoding a polypeptide with HIPHUM 46 activity may be administered to a subject suffering from a condition in which HIPHUM 46 expression is down-regulated, such as Alzheimer's disease, HIV infection and HSV infection. A nucleic acid encoding a variant of HIPHUM 46 that inhibits HIPHUM 46 activity may be administered to a patient suffering from a condition in which HIPHUM 46 expression is up-regulated such as Alzheimer's disease, myotonic dystrophy (MD), parasupranuclear palsy (PSP), Huntington's disease and amyotrophic lateral sclerosis (ALS) and HBV infection. Nucleic acid, such as RNA or DNA, and preferably, DNA, is provided in the form of a vector, such as the polynucleotides described above, which may be expressed in the cells of the mammal.

Nucleic acid encoding the polypeptide may be administered by any available technique. For example, the nucleic acid may be introduced by needle injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

The following Examples illustrate the invention.

### EXAMPLE 1

### Characterisation of the Sequence

A serine protease, designated as HIPHUM 46 has been identified. The nucleotide and amino acid sequences of the receptor have been determined. These are set out below in SEQ ID NOs: 1 and 2. Suitable primers and probes were designed and used to analyse tissue expression. HIPHUM 46 was found to be primarily expressed in cerebellum, jejunum, lung, rectum and testis. HIPHUM 46 was found to be upregulated in frontal cortex from Alzheimer's disease, myotonic dystrophy (MD), parasupranuclear palsy (PSP) and amyotrophic lateral sclerosis (ALS) brain, in parietal cortex from PSP, Huntington's disease and ALS brain, bFGF treated endothelial cells, peripheral blood mononuclear cells (PBMCs) and in HBV infection. HIPHUM 46 was found to be downregulated in cerebellum from Alzheimer's brain, in HIV infection and in HSV infection.

The chromosomal localization was also mapped. Human HIPHUM 46 has been mapped to 15q21-q22. This locus is linked to diseases such as anaemia (dyserythropoietic congenital type III), dyslexia and ceroid lipofuscinosis and is syntenic with mouse regions that are linked to diseases such as neuronal ceroid lipofuscinosis and susceptibility to experimental allergic encephalomyelitis 9.

### EXAMPLE 2

### Screening for Substances which Exhibit Protein Modulating Activity

Preparations of a purified HIPHUM 46 polypeptide or a variant are generated for screening purposes. 96 and 384 well plate, high throughput screens (HTS) are employed using fluorescence or colourimetric indicator molecules. Secondary screening involves the same technology. Tertiary screens involve the study of modulators in rat, mouse and guinea-pig models of disease relevant to the target.

A brief screening assay protocol is as follows:—

Plate Preparation for Determination of inhibitory Potency at a Single Concentration of Test Compound: Test compounds are placed in columns 1-10 of a 96-well plate. The average enzymatic activity, from the wells in column 11, is used as the control value for calculating percent inhibition. In most cases assay plates contain 1 μL/well of test compound. The control well (column 11) contains 1 μL/well of solvent (100% DMSO). For assays conducted in 384-well plates, 1 μL of test compound is added to columns 1-20 and the same volume of 100% DMSO is added to column 21. The average enzymatic activity, from the wells in column 21, is used as the control value for calculating percent inhibition.

Assay: Due to the inherent stability of the enzyme at room temperature and the low concentration of enzyme used, the enzyme, substrate and buffer are combined prior to addition to the assay plate containing test compounds. Both the stock solution of enzyme, 0.2 mg/mL, and the stock solution of substrate, 500 mM, are diluted with the solution of assay buffer to yield 3.3 ng/mL enzyme and 100 μM substrate in 25 mM Tris, pH 7.5, 10 mM KCl, 140 mM NaCl. A volume of 100 μL is added to the 96-well assay plate or a volume of 50 μL is added to the 384-well assay plate. The absorbance is monitored every 3.7 hours for 18.5 hours at 405 nm using a Wallac Victor filter-based plate reader coupled with a Zymark Twister. A maximum of 50 assay plates are monitored at one time using this automated system in order to have the 3.7 hour cycle per plate. An empty plate is added at the top and bottom of each of the 3 stacks of plates to reduce evaporation in the plates that would be exposed to air during the run. Data for a single plate at a single time point is stored in each file.

1. A method for identification of a substance that modulates serine protease activity, which method comprises:
(i) contacting
(a) a cell selected from a neuronal cell, a lung cell, an intestinal cell and a cell infected with a virus, which cell expresses a serine protease polypeptide comprising the amino acid sequence of SEQ ID NO: 2 or a variant thereof which has dipeptidyl peptidase activity with
(b) a test substance; and
(ii) monitoring for serine protease activity.

2. A method for identification of a substance that modulates serine protease activity, which method comprises:
(i) contacting
(a) a serine protease polypeptide isolated from a cell selected from a neuronal cell, a lung cell, an intestinal cell and cell infected with a virus, which serine protease comprises the amino acid sequence of SEQ ID NO: 2 or a variant thereof which has dipeptidyl peptidase activity; with
(b) a test agent; and
(ii) monitoring for serine protease activity.

3. A method according to claim 1, wherein the neuronal cell is a cerebellar cell.

4. A method according to claim 1, wherein the neuronal cell is from Alzheimer's, myotonic dystrophy, parasupranuclear palsey, Huntington's or amyotrophic lateral sclerosis brain.

5. A method according to claim 1, wherein the virus is HBV.

6. A method according to claim 1, wherein the intestinal cell is a jejunuim cell or a rectum cell.

7. A method according to claim 1, wherein the variant has at least 80% sequence identity to SEQ ID NO: 2.

8. A modulator of serine protease activity identified by a method according to claim 1.

9. A method according to claim 2, wherein the neuronal cell is a cerebellar cell.

10. A method according to claim 2, wherein the neuronal cell is from Alzheimer's, myotonic dystrophy, parasupranuclear palsey, Huntington's or amyotrophic lateral sclerosis brain.

11. A method according to claim 2, wherein the virus is HBV.

12. A method according to claim 2, wherein the intestinal cell is a jejunuim cell or a rectum cell.

13. A method according to claim 2, wherein the variant has at least 80% sequence identity to SEQ ID NO: 2.

14. A modulator of serine protease activity identified by a method according to claim 2.

15. A method of treating a subject suffering from a disease selected from a musculoskeletal disease, a HBV disease, Alzheimer's disease, parasupranuclear palsy, myotonic dystrophy, Huntington's disease and amyotrophic lateral sclerosis, which method comprises administering to said subject an effective amount of a substance which modulates proteinase activity of a polypeptide with the amino acid sequence shown in SEQ ID NO: 2 or a variant thereof.

16. A method for treating a disease selected from a musculoskeletal disease, a HBV disease, Alzheimer's disease, parasupranuclear palsy, myotonic dystrophy, Huntington's disease and amyotrophic lateral sclerosis, which method comprises:
(i) identifying a substance that modulates serine protease activity and/or expression
(ii) administering an effective amount of the substance to a patient suffering from a disease selected from a musculoskeletal disease, a HBV disease, Alzheimer's disease, parasupranuclear palsy, myotonic dystrophy, Huntington's disease and amyotrophic lateral sclerosis.

17. A method according to claim 16 wherein step (i) comprises:
(a) contacting a test substance and a serine protease polypeptide comprising
(i) the amino acid sequence of SEQ ID NO: 2 or
(ii) a variant thereof which has dipeptidyl peptidase activity or
(iii) a fragment of (i) or (ii) which has dipeptidyl peptidase activity, and
(b) determining the effect of the test substance on the activity of the polypeptide, thereby determining whether the test substance modulates serine protease activity.


SEQUENCE LISTING (2 sequences)

SEQ ID NO: 1 - DNA, 2649 bp, Homo sapiens
  CDS (1)..(2649)
atggcagcagcaatggaaacagaacagctgggtgttgagatatttgaaactgcggactgtgaggagaatattgaatcacaggatcggcctaaattggagcctttttatgttgagcggtattcctggagtcagcttaaaaagctgcttgccgataccagaaaatatcatggctacatgatggctaaggcaccacatgatttcatgtttgtgaagaggaatgatccagatggacctcattcagacagaatctattaccttgccatgtctggtgagaacagagaaaatacactgttttattctgaaattcccaaaactatcaatagagcagcagtcttaatgctctcttggaagcctcttttggatctttttcaggcaacactggactatggaatgtattctcgagaagaagaactattaagagaaagaaaacgcattggaacagtcggaattgcttcttacgattatcaccaaggaagtggaacatttctgtttcaagccggtagtggaatttatcacgtaaaagatggagggccacaaggatttacgcaacaacctttaaggcccaatctagtggaaactagttgtcccaacatacggatggatccaaaattatgccctgctgatccagactggattgcttttatacatagcaacgatatttggatatctaacatcgtaaccagagaagaaaggagactcacttatgtgcacaatgagctagccaacatggaagaagatgccagatcagctggagtcgctacctttgttctccaagaagaatttgatagatattctggctattggtggtgtccaaaagctgaaacaactcccagtggtggtaaaattcttagaattctatatgaagaaaatgatgaatctgaggtggaaattattcatgttacatcccctatgttggaaacaaggagggcagattcattccgttatcctaaaacaggtacagcaaatcctaaagtcacttttaagatgtcagaaataatgattgatgctgaaggaaggatcatagatgtcatagataaggaactaattcaaccttttgagattctatttgaaggagttgaatatattgccagagctggatggactcctgagggaaaatatgcttggtccatcctactagatcgctcccagactcgcctgcagatagtgttgatctcacctgaattatttatcccagtagaagatgatgttatggaaaggcagagactcattgagtcagtgcctgattctgtgacgccactaattatctatgaagaaacaacagacatctggataaatatccatgacatctttcatgtttttccccaaagtcacgaagaggaaattgagtttatttttgcctctgaatgcaaaacaggtttccgtcatttatacaaaattacatctattttaaaggaaagcaaatataaacgatccagtggtgggctgcctgctccaagtgatttcaagtgtcctatcaaagaggagatagcaattaccagtggtgaatgggaagttcttggccggcatggatctaatatccaagttgatgaagtcagaaggctggtatattttgaaggcaccaaagactcccctttagagcatcacctgtacgtagtcagttacgtaaatcctggagaggtgacaaggctgactgaccgtggctactcacattcttgctgcatcagtcagcactgtgacttctttataagtaagtatagtaaccagaagaatccacactgtgtgtccctttacaagctatcaagtcctgaagatgacccaacttgcaaaacaaaggaattttgggccaccattttggattcagcaggtcctcttcctgactatactcctccagaaattttctcttttgaaagtactactggatttacattgtatgggatgctctacaagcctcatgatctacagcctggaaagaaatatcctactgtgctgttcatatatggtggtcctcaggtgcagttggtgaataatcggtttaaaggagtcaagtatttccgcttgaataccctagcctctctaggttatgtggttgtagtgatagacaacaggggatcctgtcaccgagggcttaaatttgaaggcgcctttaaatataaaatgggtcaaatagaaattgacgatcaggtggaaggactccaatatctagcttctcgatatgatttcattgacttagatcgtgtgggcatccacggctggtcctatggaggatacctctccctgatggcattaatgcagaggtcagatatcttcagggttgctattgctggggccccagtcactctgtggatcttctatgatacaggatacacggaacgttatatgggtcaccctgaccagaatgaacagggctattacttaggatctgtggccatgcaagcagaaaagttcccctctgaaccaaatcgtttactgctcttacatggtttcctggatgagaatgtccattttgcacataccagtatattactgagttttttagtgagggctggaaagccatatgatttacagatctatcctcaggagagacacagcataagagttcctgaatctggagaacattatgaactgcatcttttgcactaccttcaagaaaaccttggatcacgtattgctgctctaaaagtgatataa

SEQ ID NO: 2 - protein, 882 aa, Homo sapiens
MAAAMETEQLGVEIFETADCEENIESQDRPKLEPFYVERYSWSQLKKLLADTRKYHGYMMAKAPHDFMFVKRNDPDGPHSDRIYYLAMSGENRENTLFYSEIPKTINRAAVLMLSWKPLLDLFQATLDYGMYSREEELLRERKRIGTVGIASYDYHQGSGTFLFQAGSGIYHVKDGGPQGFTQQPLRPNLVETSCPNIRMDPKLCPADPDWIAFIHSNDIWISNIVTREERRLTYVHNELANMEEDARSAGVATFVLQEEFDRYSGYWWCPKAETTPSGGKILRILYEENDESEVEIIHVTSPMLETRRADSFRYPKTGTANPKVTFKMSEIMIDAEGRIIDVIDKELIQPFEILFEGVEYIARAGWTPEGKYAWSILLDRSQTRLQIVLISPELFIPVEDDVMERQRLIESVPDSVTPLIIYEETTDIWINIHDIFHVFPQSHEEEIEFIFASECKTGFRHLYKITSILKESKYKRSSGGLPAPSDFKCPIKEEIAITSGEWEVLGRHGSNIQVDEVRRLVYFEGTKDSPLEHHLYVVSYVNPGEVTRLTDRGYSHSCCISQHCDFFISKYSNQKNPHCVSLYKLSSPEDDPTCKTKEFWATILDSAGPLPDYTPPEIFSFESTTGFTLYGMLYKPHDLQPGKKYPTVLFIYGGPQVQLVNNRFKGVKYFRLNTLASLGYVVVVIDNRGSCHRGLKFEGAFKYKMGQIEIDDQVEGLQYLASRYDFIDLDRVGIHGWSYGGYLSLMALMQRSDIFRVAIAGAPVTLWIFYDTGYTERYMGHPDQNEQGYYLGSVAMQAEKFPSEPNRLLLLHGFLDENVHFAHTSILLSFLVRAGKPYDLQIYPQERHSIRVPESGEHYELHLLHYLQENLGSRIAALKVI